(19) United States
(12) Patent Application Publication
(10) Pub. No.: US 2018/0169435 A1
(43) Pub. Date: Jun. 21, 2018

(54) Methods of Treating a Healthy Brain Using Light Therapy

(71) Applicant: Erchonia Corporation, Melbourne, FL (US)

(72) Inventor: Steven C. Shanks, Melbourne, FL (US)

(73) Assignee: Erchonia Corporation, Melbourne, FL (US)

(21) Appl. No.: 15/696,083

(22) Filed: Sep. 5, 2017

Related U.S. Application Data
(63) Continuation-in-part of application No. 15/604,363, filed on May 24, 2017.
(60) Provisional application No. 62/435,326, filed on Dec. 16, 2016.

(51) Int. Cl. A61N 5/06

(52) U.S. Cl. CPC A61N 5/0622; A61N 2005/067; A61N 5/0618

(57) ABSTRACT

Light energy is applied externally to the head of a patient who has a healthy brain to activate portions of the brain. The light is applied to the patient's scalp all over the patient's head or to desired portions of the scalp to activate desired portions of the brain. The treatment can be enhanced by activating the cranial nerves while the light is applied. The wavelengths of the applied light range from about 400-760 nm. In a preferred embodiment the applied light is about 640 nm. In a preferred embodiment, the applied light energy is applied with a pulse frequency or frequencies that mimic healthy brain function of alpha, beta, delta, and theta waves. The pulse frequencies can be applied in series, alternately, or simultaneously. The light can be emitted from a single light emitter or from multiple emitters. Preferably the applied light energy is laser light.

### CROSS-REFERENCE TO RELATED APPLICATIONS

This application claims the benefit of co-pending U.S. Provisional Application No. 62/435,326 filed Dec. 16, 2016 and is a continuation-in-part of U.S. patent application Ser. No. 15/604,363 filed May 24, 2017.

### FIELD OF INVENTION

This invention relates generally to treating brains using light therapy. This invention relates more particularly to methods for treating healthy brains to activate portions and delay the onset of brain disease.

### BACKGROUND

Neurodegenerative diseases occur when nerve cells in the brain or peripheral nervous system lose function over time and may ultimately die. Neurodegenerative disease is a broad category of brain diseases including autism spectrum disorder; Alzheimer's disease; amyotrophic lateral sclerosis (“ALS”); Creutzfeldt-Jakob disease; vascular dementia; Lewy body dementia; fronto-temporal dementia; multi-infarct dementia; vitamin B-12 deficiency syndrome; hypothyroidism; Huntington's disease; Parkinson's disease; normal pressure hydrocephalus; and tauopathies. Many types of neurodegenerative disease are progressive, in which symptoms gradually worsen over time, and can be fatal. Many of these brain diseases involve inflammation and the body's overall inflammatory response.

Neurodegenerative disease is a common problem in older demographics, causing sufferers to have significant cognitive decline with accompanying increase in cost of care and burden on caregivers. With an ageing population, the problem is likely to worsen. The causes of neurodegenerative diseases are not well known and although there are many studies underway for the treatment of the disease and its symptoms, there is no cure. Current available medications treat the symptoms, but often have unwanted side-effects. Brain changes in neuro-degenerative conditions have been shown on imaging studies to appear many years before symptom appearance and diagnosis. Understanding these changes is key to developing ways to intervene before irreversible damage has been done. It would be desirable to maintain a healthy brain and to delay the onset of neurodegenerative diseases. Ideally, a simple treatment could prevent them from occurring.

The brain also suffers from psychiatric disorders, such as depression and anxiety. Current prescription drugs for psychiatric disorders are not generally regarded very highly by the medical profession or by patients, because many of these drugs perform little better than placebos and have unwanted side-effects. It would also be desirable to maintain a healthy brain and delay or prevent depression and anxiety.

Low-level laser therapy (“LLLT”) has been shown through numerous clinical studies and regulatory clearances to be a safe and effective, simple, non-invasive and side-effect free alternative to medication and surgical procedures for the reduction of symptoms in a variety of conditions. LLLT reduces edema, improves wound healing, and relieves pain of various etiologies. It is also used in the treatment and repair of injured muscles and tendons. Application of LLLT has been shown to have the potential to alter cellular metabolism to produce a beneficial clinical effect. Based on its ability to modulate cellular metabolism and alter the transcription factors responsible for gene expression, LLLT has been found to alter gene expression, cellular proliferation, intra-cellular pH balance, mitochondrial membrane potential, generation of transient reactive oxygen species and calcium ion level, proton gradient and consumption of oxygen. LLLT stimulation of the mitochondria via low-energy light has been shown to provoke a dynamic shift in the function of an individual cell. Laser therapy has been shown to stimulate cell regeneration and later gene expression.

Experts in LLLT have long stated that the proper wavelength of light must be used to trigger the desired photobiomodulation. It is a long-held belief by experts in the field that only long, near infrared wavelengths can penetrate deep enough into a patient's tissue or bone to affect cellular behavior, and that shorter wavelengths cannot do so. For example, at least one study has shown that red light does not penetrate a patient's skull. This has led the medical and research communities to believe that the brain cannot be successfully treated with LLLT.

Electrophysiology is the study of the electrical properties of biological cells and tissues. It involves measurements of voltage changes or electric current on a wide variety of scales from single ion channel proteins to whole organs like the heart. In neuroscience, it includes measurements of the electrical activity of neurons and, in particular, action potential activity. Recordings of large-scale electric signals from the nervous system, such as electroencephalography, may also be referred to as electrophysiological recordings. They are useful for electrodiagnosis and monitoring. For example, electroencephalography is commonly used to diagnose brain diseases such as epilepsy.

To determine the location of the brain activity with a resolution greater than what is provided by scalp electroencephalography, neurosurgeons may implant electrodes or insert penetrating depth electrodes under the dura mater, using either a craniotomy or a burr hole. The recording of these sub-dural signals is referred to as intracranial electroencephalography. The signal recorded from intracranial electroencephalography is on a finer scale of activity than the brain activity recorded from scalp electroencephalography. Low voltage, high frequency components that cannot be seen easily (or at all) in scalp electroencephalography can be seen clearly in intracranial electroencephalography. Penetrating microelectrodes are also used to determine the location of brain activity. Quantitative electric tomography is another technique to determine the location of brain activity which combines anatomical information of the brain by MRI with electroencephalography patterns, to estimate the location of the electrical activity within the brain.

While electroencephalography measures the brain's electrical activity directly, other methods measure the electrical activity indirectly. For example, single-photon emission computed tomography (“SPECT”) and functional magnetic resonance imaging (“fMRI”) record changes in brain blood flow, which is directly correlated to brain activity. Positron emission tomography (“PET”) and near-infrared spectroscopy (“NIRS”) measure metabolic activity in the brain, which are also directly correlated to brain activity.

It is an object of this invention to provide a non-invasive method of activating portions of a healthy brain, thereby maintaining a healthy brain, delaying the onset of brain diseases and disorders, or preventing them entirely.

### SUMMARY OF THE INVENTION

Light energy is applied externally to the head of a patient who has a healthy brain to activate portions of the brain. Preferably the light is applied to the patient's scalp all over the patient's head, but may also be applied to desired portions of the scalp to activate desired portions of the brain. Due to the systemic effects of applying light anywhere to the brain, the more areas of the head that can be treated the more effective treatment is. The treatment can be enhanced by activating the cranial nerves while the light is applied.

The wavelengths of the applied light range from about 400-760 nm. In a preferred embodiment the applied light is about 640 nm. In a preferred embodiment, the applied light energy is applied with a pulse frequency or frequencies that mimic healthy brain function of alpha, beta, delta, and theta waves. The pulse frequencies can be applied in series, alternately, or simultaneously. The light can be emitted from a single light emitter or from multiple emitters. Preferably the applied light energy is laser light.

### BRIEF DESCRIPTION OF THE DRAWINGS

FIG. 1 illustrates a portable, floor-supported light-emitting device for treating a patient's head.

FIG. 2 illustrates the light-emitting device of FIG. 1 employing a translucent cap around a patient's head.

FIG. 3 illustrates a wall mounted light-emitting device for treating a patient's head.

FIG. 4 is a schematic illustration of a brain inside a patient's skull.

FIG. 5 is a schematic illustration of a brain.

FIG. 6 is a prior-art table of cranial nerves.

FIG. 7 shows QEEGT images within the gamma band of a healthy brain before and after the application of laser energy.

### DETAILED DESCRIPTION OF THE INVENTION

This is a non-invasive method of activating portions of a healthy brain, thereby maintaining a healthy brain, delaying the onset of cognitive decline, brain diseases and disorders, or preventing them entirely. A healthy brain, as used herein, means a brain that shows no measurable signs or symptoms of cognitive decline, neurodegenerative disease or damage.

The method involves applying an effective amount of light energy to the patient's head. By applying light energy, portions of the brain are activated. To “activate” as used herein means to change the electrical state of cells or tissues, in a positive or negative direction. That is, activation can refer to both increasing and decreasing electric current, voltage, potential or magnetic fields of cells or tissues. The activation may, in turn, open or close ion channels, cause the transition of one molecule into another state, convert biological molecules from a passive state to an active state or vice versa, and thereby modulate brain function. The activation may be temporary, reversible or permanent.

The activation may be measured by direct methods such as electroencephalography, either on the scalp or intracranially, both referred to herein as EEG unless expressly differentiated. The activation may be measured by indirect methods such as SPECT, fMRI, PET, NIRS, or a combination of direct and indirect methods. The activation may be more clearly quantified by combining measurements of brain activation with other anatomical information, such as electric tomography and electrooculogram.

There are a number of variables in light therapy, including the wavelength of the light, the power of the light source, the pulse frequency, the area impinged by the light, the shape of the beam spot when the light impinges the treated area, the intensity or fluence of the light energy, and the treatment duration. The setting of these variables typically depends on the brain, skull, and tissue characteristics of the specific patient and, in cases in which a patient has a propensity for a specific disease or disorder, areas of the brain to be treated. The success of each therapy depends on the relationship and combination of these variables. For example, the brain may be treated to delay Alzheimer's disease with one regimen utilizing a given power, wavelength, pulse frequency and treatment duration, whereas the brain may be treated to delay depression with a regimen utilizing a different power, wavelength, pulse frequency and treatment duration, and either regimen may be further adjusted for a given patient depending on that patient's size, weight, and age.

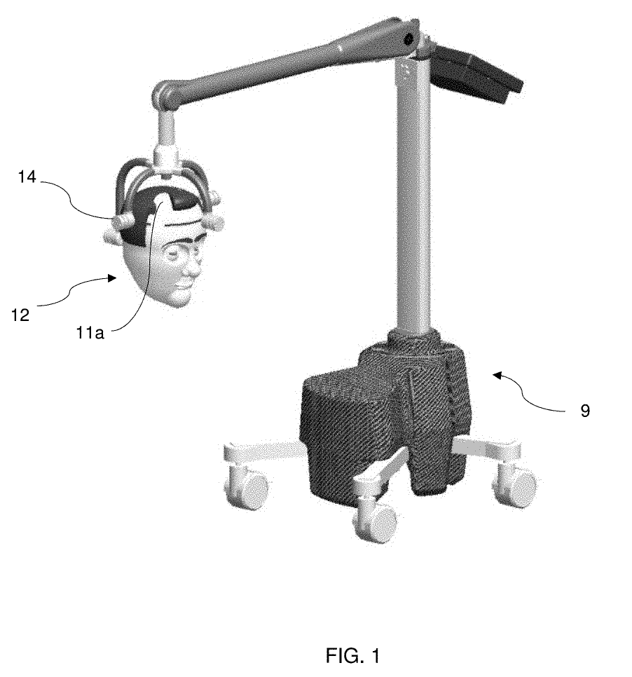

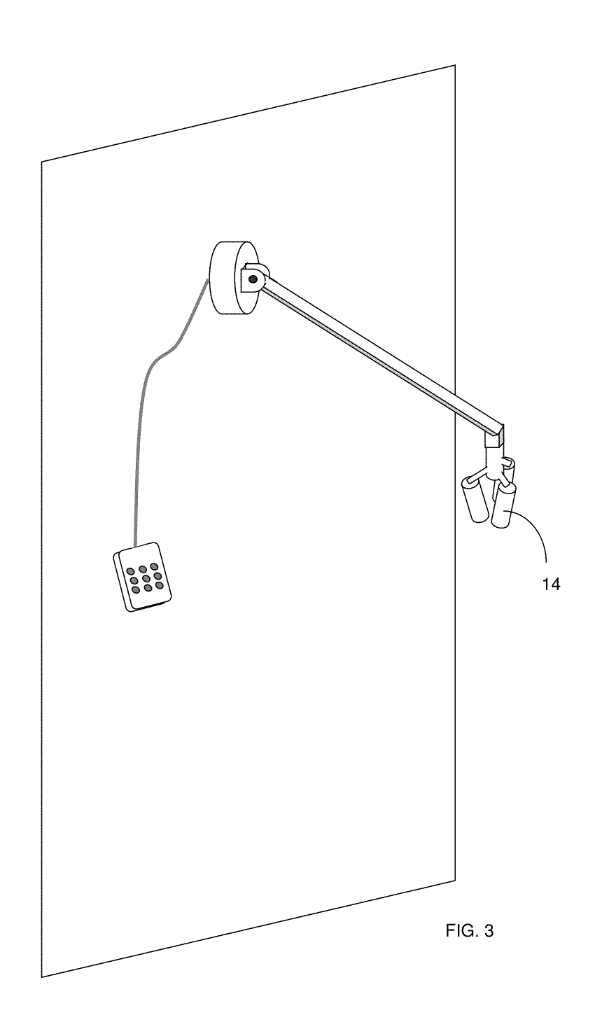

The wavelengths of the light that can be applied range from about 400-1200 nm nominal, with the desired wavelength within the spread from nominal. In some embodiments multiple wavelengths are used, either in series, alternately, or simultaneously. In a preferred embodiment, the applied light has a wavelength in the red range, and more preferably at 640 nm nominal. The light can be from any source including light-emitting diodes, hard-wired lasers, or laser diodes, but preferably is from a semiconductor laser diode such as Gallium Aluminum Arsenide (GaAlAs) laser diodes, emitting red laser light at 640 nm nominal. Commercial semiconductor laser diodes have a spread of ±10 nm from nominal so the light applied is within the spread from nominal. FIGS. 1, 2, 3 and 5 illustrate probes 14, each containing a laser diode.

The applied light is emitted from emitters having a power output of 1 mw to 20 watts. In one embodiment the emitted light is low-level light therapy has an energy dose rate that causes no immediate detectable temperature rise of the treated tissue and no macroscopically visible changes in tissue structure. Consequently, the scalp tissue impinged by the light, the skull, and the brain and nerve tissue are not heated and are not damaged. In another embodiment, the energy dose rate causes a detectable warming of the tissue, sometimes with a concomitant flush to the skin due to the response of the nervous system leading to widening of the capillaries of the involved skin, but in no embodiment is the tissue damaged. The energy emitted from the light devices may range from 0.0001 to 1500 joules.

The applied light energy is applied with a pulse frequency or frequencies of brain waves emanating from a healthy brain, as measured by electroencephalography. Brain waves are neural oscillations in a rhythmic or repetitive neural activity that includes the following:

| Wave | Approximate Frequency | Main Source Location |
|---|---|---|
| Type | Range in Humans | on the Human Brain |
| Delta | 0.5 to 3 Hz 0.5-4 | thalamus or cortex |
| Theta | 3 to 8 Hz | hippocampus |
| Alpha | 8-12.5 Hz | occipital lobe |
| Mu | 7.5-12.5 (and primarily 9-11) | motor cortex |
| | Hz | |
| Beta | 12.5 to 38 Hz | posterior brain |
| Gamma | 38 to 100 Hz | all areas of brain |

Other types of oscillatory activity are found in a healthy central nervous system, and light therapy may be applied at a pulse frequency that mimics that oscillatory activity. Multiple pulse frequencies can be applied series, alternately, or simultaneously. In one embodiment, the light therapy is applied using several light sources, each having a different frequency.

The method is non-invasive. Light energy is applied externally to the head 11 of a patient who has a healthy brain to activate portions of the brain. The light may be applied to a patient's shaved skull 11a, through the patient's hair, or through a translucent skull cap 20 which may also aid in orienting the light to the desired location on the patient's head. Typically the patient is treated while the patient is vertical or nearly vertical, as opposed to prone or supine, so that all regions of the skull and brain stem can be treated without moving the patient. The patient can be awake, sedated, or asleep.

Preferably the light is applied to the patient's scalp all over the patient's head, but may also be applied to desired portions of the scalp to activate desired portions of the brain. In some cases it may be desirable to activate only portions of the brain. The frontal portion of the brain plays a direct role in behavior, intelligence memory and movement. The parietal portion plays a direct role in intelligence, language, reading and sensation. The cerebellum plays a direct role in balance, swallowing, breathing and heartbeat. The temporal portion plays a direct role in speech, vision, hearing, and long-term memory. Due to systemic effects of applying light anywhere to the brain, the application of light on any area of the skull will activate portions of the brain, and application to multiple areas is often beneficial. All portions of the brain may be treated, alone or in combination with other portions. In a preferred embodiment, the entire head of a patient except the face is treated with light energy. In another embodiment, the light energy is applied to the frontal lobe, occiput, cerebellum, cortex, and brain stem. In one embodiment the treatment is applied to a specific hemisphere of the brain.

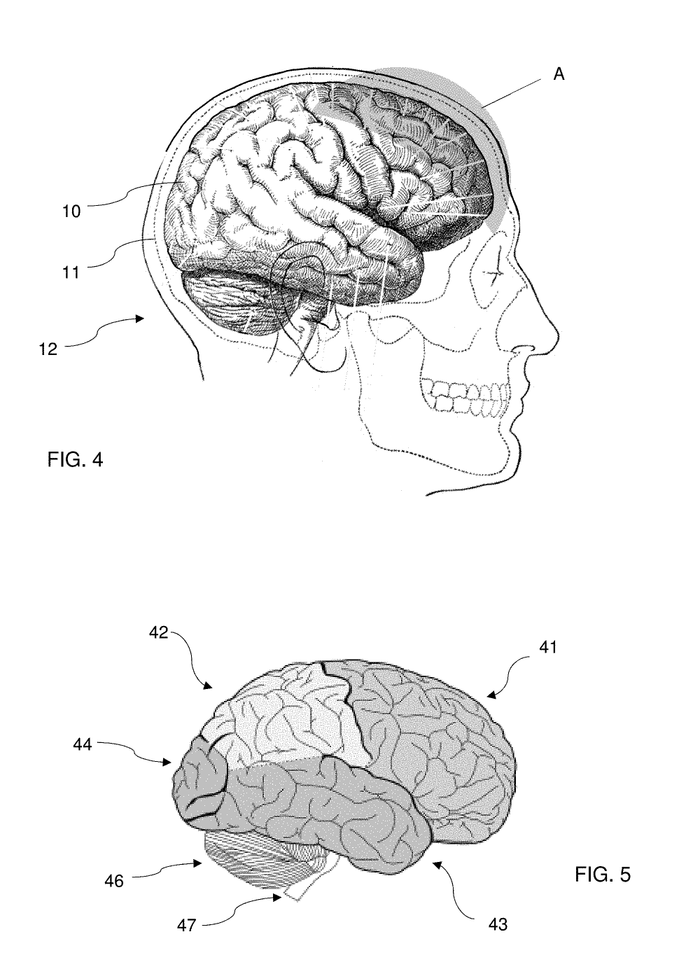

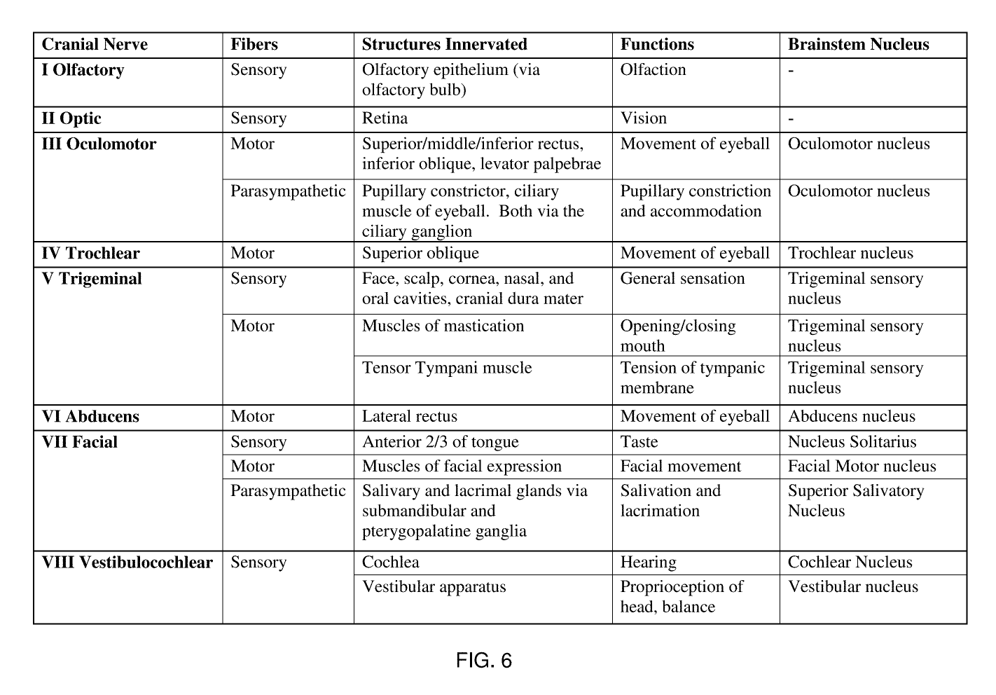

As used herein, light applied “to” or “near the” area means light applied to the scalp at a position mapped to the area of the brain to be treated, such as the frontal 41, parietal 42, temporal 43, and occipital 44 lobes; the cortex; cerebellum; the brain stem; or where one or more cranial nerves enters the brain. See FIG. 5. For example, if the light is to be applied “to the frontal cortex,” it will be applied to the scalp above the frontal cortex, as indicated generally by area A in FIG. 4. In another example, if the light is to be applied “near the area” of the basal ganglia, which is in the center of the brain, the light will be applied to the scalp all around the head from about the ear lobes up to the top of the head.

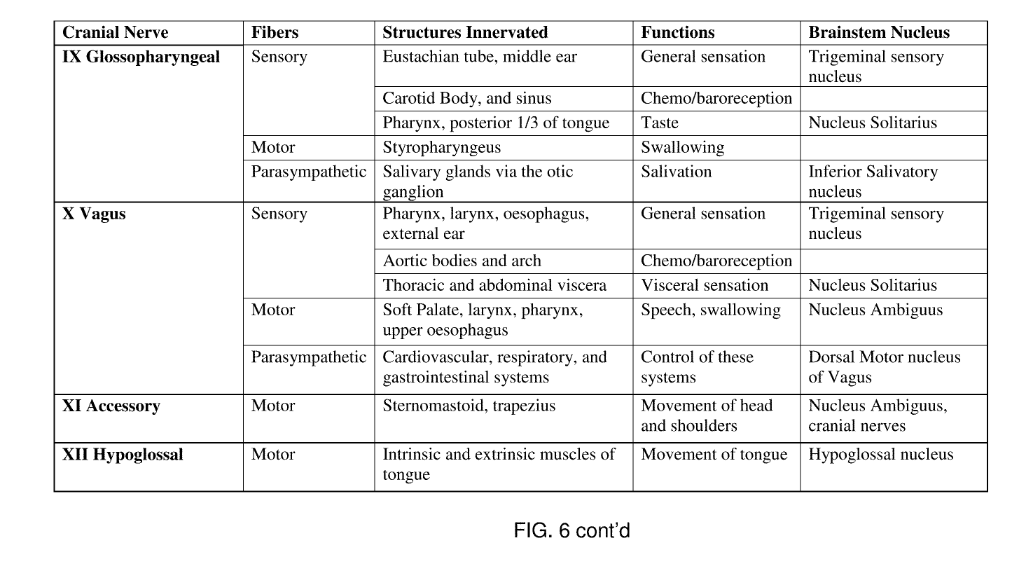

The treatment can be enhanced by activating the cranial nerves while the light is applied. FIG. 6 is a table of the cranial nerves and their functions. A cranial nerve is activated by having the patient to execute the function indicated the table of FIG. 6. For example, to activate the olfactory nerve, the patient would be given something with an odor or scent to smell. Similarly, to activate the trochlear nerve, the patient would move his eyeballs. Due to the systemic effects of applying light anywhere to the brain, the application of light on any area of the skull will work on any malady to some degree, and application to multiple areas is often beneficial. In one embodiment the treatment is applied to a specific hemisphere of the brain. For some diseases the treatment is applied to acupuncture points on the brain. The light may be applied in stationary manner relative to the skull, or it may be scanned across the skull. The light may be provided by a single light source or multiple light sources. U.S. Pat. No. 7,118,588, also incorporated here by reference, discloses a line generator for laser light application. At 4 inches away from the scalp, the 70 degree line generator as disclosed therein creates a projected beam that is 14 cm long and 3 mm wide for a total beam area of 420 mm². The size of each treatment area is the same as the beam profile because the diode is not moved during procedure administration. Given a 600 second treatment time with a 7.5 mW laser, the total energy each produced by each independent laser diode is 4.5 joules per laser. The fluence per laser is calculated as 0.011 J/cm². Using a laser with 5 independent laser diodes, as shown in FIG. 1, each laser diode treats a different (separate) areas of the head, so the fluence remains the same (unchanged) at 0.011 joules; however, the total energy delivered to the subject per 10-minute procedure administration across all 5 laser diodes and respective treatment areas combined is 22.5 joules.

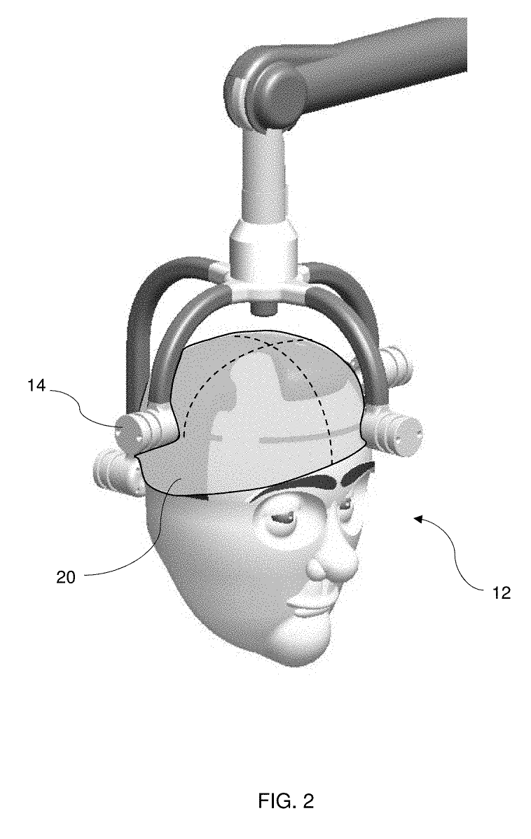

The light can be applied using a variety of light emitting devices, including an array of LEDs in a rigid or flexible wrap or helmet, a hand-held LED or laser device, a full-body LED or laser scanner, a wall-mounted LED or laser device, or a stand-alone LED or laser device. Handheld lasers are described in U.S. Pat. Nos. 6,013,096 and 6,746,473, which are incorporated herein by reference. A full-body laser scanner is described in U.S. Pat. No. 8,439,959, incorporated herein by reference. Wall-mount and stand-alone lasers 9 are described in U.S. Pat. No. 7,947,067 as illustrated in FIGS. 1 and 2 and incorporated herein by reference. The light source may be battery-powered or connected to mains power.

In a preferred embodiment the shape of the beam spot on the treated area is an apparent circle, which is actually a rotating diameter by a line of light. U.S. Pat. No. 7,922,751, incorporated herein by reference, discloses a device to sweep such a circular beam spot. The device disclosed in that patent can be programmed to move the scanning head in a manner to achieve any desired shape of a treatment zone on the head of a patient. A sample selection of available scan patterns is shown in that patent at FIGS. 8a-h.

A therapeutically effective amount of light energy is applied to the brain to activate at least a portion of the brain. The duration of each treatment may range from one second to 30 minutes. In a preferred embodiment, the therapeutically effective amount is 10 minutes. Treatments may be repeated periodically to activate at least a portion of the brain each time and maintain brain health.

Example of Activation

A 28-year old patient with a healthy brain was treated in a room with controlled temperature from 24 to 26 C, noise attenuation, and dimmed lights. Twenty minutes of EEG were recorded immediately before treatment. Laser light was applied for 10 minutes to the patient's skull through his hair using a hand-held laser. The light energy was applied by the Erchonia® EAL Laser, a hand-held laser with two 640 nm nominal semiconductor laser diodes at pulse frequencies of 4 Hz, 12 Hz, 33 Hz, and 60 Hz. The light energy was applied to the frontal lobe, occiput, cerebellum, cortex, and brain stem using a sweeping motion continuously during treatment. Twenty minutes of EEG were recorded immediately after treatment.

The data were assessed using quantitative EEG and quantitative electric tomography (“QEEGT”). QEEGT is a technique that combines anatomical information of the brain by MRI with EEG patterns, to estimate the sources of the EEG within the brain. The EEG was recorded using nineteen monopolar derivations of the International 10-20 System (FP1, FP2, F3, F4, C3, C4, P3, P4, O1, O2, F7, F8, T3, T4, T5, T6, Fz, Cz, Pz) with linked earlobes as a reference. Eye movement artifacts were monitored by use of the electrooculogram (EOG). The data acquisition was performed using a MEDICID-07 System (Neuronic, S.A.). After visual editing to remove artifacts, 48 artifact-free samples were selected, each 2.5 seconds long, for each experimental condition, and were transformed using the FFT to the frequency domain, yielding a power spectrum from 0.78 to 70 Hz with a sampling frequency of 0.39 Hz (178 frequencies), with a 60 Hz notch filter.

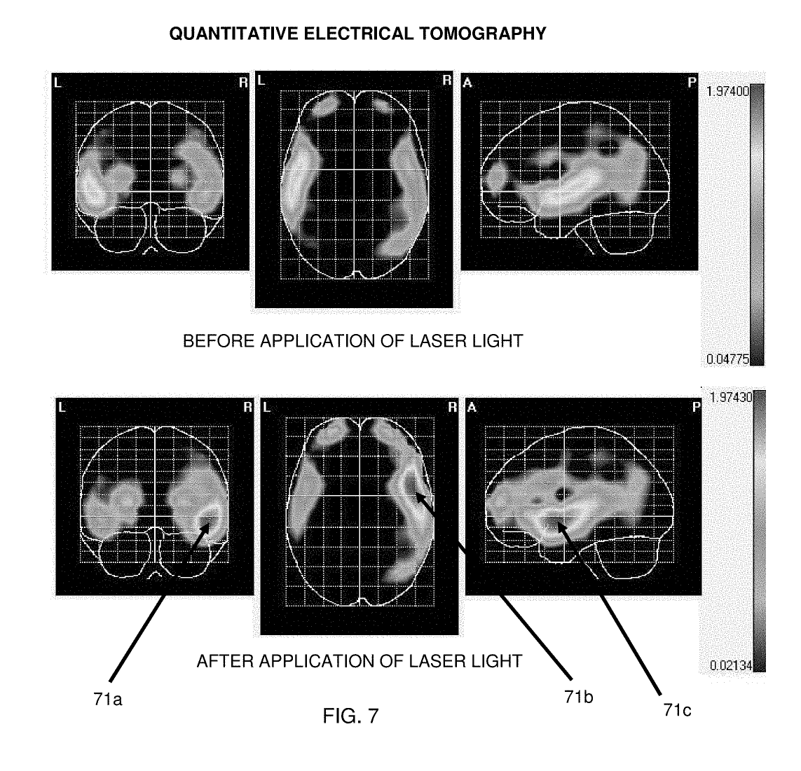

FIG. 7 shows images of the QEEGT of the patient's brain, before and after treatment. After treatment the patient showed increased brain activity within the gamma band. Arrows 71a, 71b, and 71c point to the portions of the brain having increased gamma band activity. A decrease in gamma-band activity is known to be associated with cognitive decline, and increasing the gamma band activation may help maintain a healthy brain and delay onset of cognitive decline.

While there has been illustrated and described what is at present considered to be the preferred embodiments of the present invention, it will be understood by those skilled in the art that various changes and modifications may be made and equivalents may be substituted for elements thereof without departing from the true scope of the invention. Therefore, it is intended that this invention not be limited to the particular embodiments disclosed, but that the invention will include all embodiments falling within the scope of the appended claims.

1. A method of activating a desired portion of a patient's healthy brain comprising applying an effective amount of light energy to the patient's skull above the Reid line and at a pulse frequency of brain waves emanating from a healthy brain.

2. The method of claim 1 wherein the light energy has a wavelength in the red range.

3. The method of claim 1 wherein the light energy has a wavelength of 640 nm nominal.

4. The method of claim 1 wherein the light energy is applied using a light-emitting device that is in direct contact with the patient's scalp.

5. The method of claim 1 wherein the light energy is applied using a light-emitting device that is not in direct contact with the patient.

6. The method of claim 5 wherein the light emitting device is a laser and the method further comprises scanning the emitted laser light on the patient's scalp.

7. The method of claim 9 wherein the activation comprises increased activity in the patient's brain in the range of 38 Hz-100 Hz as measured by EEG.

8. The method of claim 1 further comprising:
a. measuring the electrical activity of the desired portion of the brain before applying light energy; and
b. measuring the electrical activity of the desired portion of the brain after applying light energy.

9. The method of claim 8 wherein the measuring is done by EEG, SPECT, fMRI, PET, NIRS or a combination thereof.

10. The method of claim 1 further comprising activating one or more cranial nerves while applying the light energy.

11. The method of claim 1 wherein the light energy has a pulse frequency of brain waves emanating from a healthy brain.

12. The method of claim 1 wherein the light energy is applied at a pulse frequency of one or more of delta, theta, alpha, mu, beta or gamma brain waves.

13. A method of delaying the onset of cognitive decline in a healthy brain comprising applying light energy to one or more portions of the healthy brain above the Reid line and at a pulse frequency of brain waves emanating from a healthy brain to activate the brain.

14. The method of claim 13 wherein the light energy is emitted from a laser.

15. The method of claim 13 wherein the light energy is applied at a pulse frequency of one or more of delta, theta, alpha, mu, beta or gamma brain waves.

16. The method of claim 13 wherein the light energy has a wavelength of 640 nm nominal.

17. A therapeutic method to treat a healthy brain comprising applying a sufficient amount of light energy to the brain above the Reid line and at a pulse frequency of brain waves emanating from a healthy brain to activate at least a portion of the brain.

18. The method of claim 17 wherein the light energy is emitted from a laser.

19. The method of claim 17 wherein the light energy has a wavelength in the red range.

20. The method of claim 17 wherein the light energy has a wavelength of 640 nm nominal.
